(19) United States
(12) Patent Application Publication
(10) Pub. No.: US 2014/0113327 A1
(43) Pub. Date: Apr. 24, 2014

(54) BIOCHEMICAL SENSOR FOR QUANTITATIVE SIMULTANEOUS MULTI-SPECIES BACTERIA DETECTION IN SITU

(71) Applicant: Environmental Technology Solutions LLC, Mesa, AZ (US)

(72) Inventors: Gary M. Nijak, JR., Tempe, AZ (US); Joseph R. Geary, Alexandria, VA (US); Matthew B. Sawtelle, Mesa, AZ (US)

(73) Assignee: Environmental Technology Solutiions LLC, Mesa (US)

(21) Appl. No.: 13/993,012

(22) PCT Filed: Mar. 8, 2013
(86) PCT No.: PCT/US2013/029961

§ 371 Date: Jun. 10, 2013

(30) Foreign Application Priority Data
Mar. 9, 2012 (US) 61609041
Dec. 10, 2012 (US) 61735239

(51) Int. Cl. C12Q 1/06; C12Q 1/10

(52) U.S. Cl. CPC C12Q 1/06; C12Q 1/10
USPC 435/38; 435/39; 435/288.7

(57) ABSTRACT

Methods for detecting concentration target organisms in water. The methods involve adding a tagged reagent to a water sample, wherein the tagged reagent is water soluble; and determining a concentration of at least one bacteria in the water sample based on an intensity of an emission emitted from the water sample in response to exposure to light having a known wavelength. An apparatus including a reaction chamber; a reversible pump, a reagent source comprising a fluorophore-tagged reagent, a light source, an optical detector disposed to detect fluorescence emitted from the reaction chamber in response to light emitted from the light source; a processor configured to communicate with the reversible pump, the reagent source, the light source, and the optical detector, the processor being configured to determine the concentration of one or more target organisms in a water sample.

### CROSS-REFERENCES TO RELATED APPLICATIONS

This application claims benefit under 35 U.S.C. §119(e) to U.S. Provisional Patent Application Ser. No. 61/609,041 filed on Mar. 9, 2012, and to U.S. Provisional Patent Application Ser. No. 61/735,239 filed on Dec. 10, 2012, which are hereby incorporated by reference in their entireties.

### BACKGROUND OF THE INVENTION

1. Field of the Invention

The invention relates generally to biochemical sensors and more specifically to biochemical sensors for multi-species bacteria detection.

2. Description of the Related Art

Water quality monitoring is a recent topic of focus and has received significant research attention. Recreational waters such as ponds, lakes, streams, rivers, and beaches, used for activities such as swimming and boating are at high risk of bacterial outbreaks. The protection of recreational waters has improved as communities have increased efforts to manage and reduce waste contamination, but bacterial outbreaks remain a consistent concern. The principal methods to determine that contamination has occurred are inferential: sufficient rainfall to trigger a CSO discharge is reason to conclude that it has occurred. Sampling and lab testing often follows to confirm the magnitude of the release. Field-mount sensors to detect the presence of pathogens are not commercially available at this time. There is an ongoing effort to improve sensor design with the priorities of rapid detection and accurate readings.

Federal regulations have recently been updated as the government seeks to protect recreational users of public bodies of water. While culture-based methods are the standard for fecal contamination identification, many scientists are now recommending the switch to monitors which can provide real-time detection. Often, when there is heavy rain, combined storm/sanitary collection facilities are forced to discharge a portion of the influent without treatment to prevent backup into homes and buildings. These occurrences are referred to as Combined Sewage Overflow (CSO) discharges, and the ability for water-quality monitors to quantify their magnitude is paramount. In order to meet this demand, many different governmental, academic and industrial researchers are seeking safe, reliable, and effective sensors.

The current practice for the enumeration of bacteria in a sample involves culturing the water sample in ideal conditions in order to magnify the amount of bacteria present and to facilitate a count of colony forming units (CFUs) per 100 mL of sample water. While this method is accurate and reliable, it is time consuming and often impractical. There is little room for automation and the time required for culturing can introduce considerable lag into potential contamination response and remediation. Laboratory-grade instruments can be mounted in hardened enclosures allowing them to be brought to the field so that samples can be analyzed before being taken to a lab, but this method requires even more human intervention. A serious need exists for an improved sensor capable of enumerating indicator organisms as a measurement of the actual impact of CSO discharges on a body of water. Such a device would monitor source waters for water supply and receiving waters for treated wastewater on a continuous or intermittent basis. Results could be stored in the device, reported out via wireless communication link. The only human intervention required would be performance of routine maintenance. Market research indicates a strong desire for such a device among Municipal Water and Wastewater departments, particularly in the East Coast, West Coast, and Great Lakes areas. Significant savings in labor will result from the deployment of sensors that can automatically detect and report on the presence of Enterococcus, E. coli, and other pathogens. Regulatory compliance requires many municipalities to sample weekly for pathogens. Recreational waters and beaches are among the principal monitored areas. Often, beaches are closed preemptively, despite the high economic impact of closing, until sampling and testing can confirm that it is safe to reopen. A network of sensors that can detect pathogens would provide a cost-effective supplement to sampling programs. Demonstrated performance of such sensors would reduce the requirement for sampling while increasing the reliability and timeliness of the information generated.

In particular, there is a need for a single sensor that is capable of detecting multiple pathogens. While E. coli is the contaminant most frequently of interest, Enterococcus is considered by many to be a more reliable indicator of human waste and is therefore of concern. The device described herein is designed to address the needs of agencies and departments responsible for water and wastewater treatment and for monitoring of source and receiving waters, particularly those that have recreational elements including beaches.

### BRIEF SUMMARY OF THE INVENTION

The present invention is directed to methods and devices that can satisfy the need for pathogen detection in recreational and source waters.

A device, according to one embodiment, can automatically sample, analyze, and wirelessly transport data for a water sample to simultaneously detect multiple pathogens. The device can include pumps to collect the water sample, a reactor to mix the water sample with chemical reagents (the reagents can be selected from Table 1 below); a chemical reagent that is hydrolyzed by enzymes produced by the pathogen of interest, releasing a fluorophore; at least one light source, which can be a light emitting diode where the wavelength of the light source sufficiently excites the fluorophore near its maximum adsorption wavelength; at least one optical detector which can be either a photodiode or a charged coupled device array which can detect the emitted light from the excited fluorophore; a pump to purge the system after the assay; a controller that starts and stops each assay; a wireless component that can report results to a networked location or a data logger that collects data over an extended time period; a power component that allows the sensor to operate remotely; and a waterproof container.

According to various embodiments, the device can simultaneously detect several strains of bacteria in a water sample; can operate in all types of water including saline, brackish, fresh, and marine waters; is reusable in the field without human interaction except to exchange consumables; and can allow for automated and standardized detection of pathogens in rapid time frames (less than 24 hours).

According to one embodiment, the device can comprise a sensor that utilizes enzymes to hydrolyze a tagged sugar fluorophore complex bound by a glycosidic bond, where the enzyme is β-D-galactopyranosidase, β-D-glucopyranosidase, or β-D-glucuronidase, where one or all may be detected simultaneously. The tagged sugar fluorophore is preferably hydrophilic and water soluble as to more easily facilitate use in the automated sensor and allow for a simplified rinsing ability. The fluorophore-tagged sugar can be detected with an optical probe either in the ultraviolent wavelength, visible wavelength, infrared, or near infrared wavelength.

According to various embodiments, the intensity of the optical signal is measured over time and compared to the background signal from the detector and upon reaching a threshold the sample is quantified based upon the time to reach the predetermined threshold.

In still further embodiments, the sensor can contain a heating element to maintain the reactor system at a temperature in a range of from 70 to 120° F., preferably from 80 to 100° F., most preferably at about 98° F.

The system pumps in a sample from a body of water which is subsequently mixed with a fluorophore-tagged reagent in a reactor vessel. In the presence of pathogenic bacteria, enzymes cleave the fluorophore from the reagent. The fluorophore is subsequently excited from a controlled light source after being freed into the water. The fluorescence intensity is measured and correlated to a concentration of bacteria. The correlation can be performed using an algorithm or any other suitable method.

Methods for detecting the concentration of one or more target organisms in a water sample are also provided. The methods involve adding a tagged reagent to a water sample, wherein the tagged reagent is water soluble; and determining a concentration of at least one bacteria in the water sample based on an intensity of an emission emitted from the water sample in response to exposure to light having a known wavelength.

These and other features, aspects, and advantages of the present invention will become better understood with reference to the following description and appended claims.

### BRIEF DESCRIPTION OF THE DRAWINGS

These and other features, aspects, and advantages of the present invention will become better understood with reference to the following description and appended claims, and accompanying drawings where:

FIG. 1: is a schematic block diagram of a system according to one embodiment;

FIG. 2: is a flowchart summarizing a method according to one embodiment;

FIG. 3: is a chart showing different intensity responses to various concentrations of three monofluorophores with the same integration time (50 msec);

FIG. 4: is a chart showing the recorded intensity at 517 nm as a function of fluorescein and resorufin concentration;

FIG. 5: is a chart showing the recorded intensity at 582 nm as a function of fluorescein and resorufin concentration; and

FIG. 6: is a correlation curve showing that the time to reach the detection threshold varies with the concentration of the bacteria present in the sample.

It should be understood that the various embodiments are not limited to the arrangements and instrumentality shown in the drawings.

### DETAILED DESCRIPTION OF THE INVENTION

Many natural environments have been compromised in recent years due to the contamination arising from fecal matter. Areas downstream from sewage treatment plants and Combined Sewer Overflow (CSO) points are especially susceptible to contamination. While there are many options for handling such contamination incidents, and they are readily detectible, the appropriate reaction varies with the magnitude of the contamination and quantification of such incidents currently involves considerable time lag.

There exists a pressing need for a widely-available sensor which can provides rapid notification in the event of the presence of fecal contaminants. In addition, sensors which operate in a network will improve this tracking. In order to be able to provide consistency, sensors need to be able to account for varying levels of salinity in their measurements. Because saline environments alter bacterial growth rates and enzyme activity, these factors must be included in sensor design and data-interpretation algorithms.

A design has been developed which allows for the real-time simultaneous detection of multiple bacterial contaminants in water systems. The device is further designed to operate effectively in waters with varying degrees of salinity. As various sensors become commercially available, most will likely focus on the detection of a specific indicator organism or compound. Potential bacterial candidates for indication of fecal matter contamination include those of the general Escherichia, Enterococcus, and Streptococcus (most especially S. pneumoniae) as well as several other species of coliform bacteria. E. coli is currently the standard for fecal matter detection, but recent research has suggested that enterococci are more stable indicators of contamination. Each species is unique and requires a specially attuned sensor for detection. The ability to detect multiple potential indicators of contamination has not yet been demonstrated in an autonomous sensor.

Of greatest interest is a sensor which can work in a wide gradient of salinity. As contamination moves from fresh water to brackish water, a truly robust sensor is able to account and correct for resultant measurement alterations. Marine E. coli enumeration on an indicator-organism basis must be calculated differently than fresh water enumerations. Many receiving waters experience varying degrees of salinity depending on tides and droughts. The integration of a range of salinity conditions into sensor performance would further improve the versatility of the device in monitoring water quality.

Sensors, according to various embodiments, can be designed to meet each of these needs in a way that is unique to the current field. The activity of enzymes released by bacteria which are indicators of the presence of fecal matter can be measured by this sensor to detect a wide range of contamination levels accurately. The enzymes from bacteria have specific activity on distinct saccharide molecules. When those molecules are bound glycosidically to fluorescent moieties, enzymes are able to hydrolyze that bond and fluorophores are released into solution. An optical sensor can detect the emission wavelength of these excited fluorophores and the rate of increase in intensity is correlated through an algorithm that compares the current fluorescent intensity to a background fluorescent intensity that is selected from a wavelength that shows no fluorescence to indicator organism presence, adjusting for current water conditions.

Because there are several different organisms which can be used as indicators of fecal matter contamination, there are many ways in which a sensor may detect such contamination. Sensors, according to various embodiments, are designed for this application, and can utilize a number of different compounds to detect and differentiate between multiple enzymes. By using peak emission values as previously listed, the sensors can accurately and rapidly detect differing levels of contamination in situ. This novel coupling of biochemical and environmental engineering will provide those with stewardship over water-quality a powerful tool enabling faster response and more organism-specific protection for recreational bodies of water.

The present invention may be understood more readily by reference to the following detailed description of preferred embodiments of the invention as well as to the examples included therein. All numerical values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Various bacteria can be indicative of fecal matter. Types of bacteria indicative of the presence of fecal matter can include, but are not limited to, the genera Escherichia, Enterococcus, Streptococcus, coliform bacteria, and combinations thereof. In particular, the bacteria S. pneumonia or E. coli is indicative of human fecal matter.

Fluorophores can be released during an enzyme-catalyzed glycosidic reaction, occurring in the presence of bacteria indicative of fecal matter. A glycosidic reaction is a type of chemical reaction that involves creating or breaking at least one glycosidic bond. A glycosidic bond is a type of covalent bond that joins a carbohydrate (sugar) molecule to another group, which may or may not be another carbohydrate.

Various embodiments relate to devices comprising sensors or methods employing sensors for optically detecting fluorophores released during such a glycosidic reaction occurring in the presence of a bacteria indicative of fecal matter. Generally, a fluorophore is a fluorochrome (or fluorescent chromophore) covalently bonded to a macromolecule and used to stain tissues, cells, or materials for fluorescent imaging and spectroscopy. The fluorophore absorbs light energy of a specific wavelength and re-emits energy at a longer wavelength. The wavelength, amount, and time before emission of the emitted energy depend on both the fluorophore and its chemical environment as the molecule in its excited state interacts with surrounding molecules. Various embodiments employ a light source, such as a light emitting diode with a specific wavelength that excites a fluorophore near its maximum adsorption wavelength. Thereby, the fluorophore, if present, is prompted to re-emit energy, which can be detected by at least one optical detector.

Various embodiments can detect multiple indicator organisms (Escherichia, Enterococcus, Streptococcus, coliform bacteria) simultaneously and in situ; and possess the ability to operate in complex waters with high turbidity, and various plant and animal nutrient contents, as well as in multiple types of waters, including fresh, brackish and marine waters.

Various embodiments, therefore, provide significant advancements over currently available devices and methods. The devices and methods according to various embodiments can facilitate water quality management and control through greater autonomy provided by the automated sampling, pumping, and result-reporting components of the sensor and a broader scope of applicability. Devices and methods according to various embodiments allow for automatic sampling of waters, automatic detection of pathogens in the water sample, automatic reporting of the results via a wireless system, and much more rapid result reporting than otherwise available. Previous approaches to pathogen detection have required binding agents and have utilized non-reusable materials that require ongoing maintenance. The devices and methods according to various embodiments of the invention allow for the ongoing, low maintenance, remote, unattended reporting of results in rapid time frames. Therefore, embodiments of the invention can facilitate improved public health and sanitation, especially with respect to recreational waterways.

### Enzymes

The devices and methods, according to various embodiments, can utilize various enzymes produced by the specific bacteria strains during their metabolism. β-glucuronidase can be used in the detection of Escherichia coli. β-galactopyranosidase also known as β-galactosidase can be used in the detection of fecal coliforms. β-glucopyranosidase also known as β-glucosidase can be used in the detection of enterococci.

The enzymes that the bacteria produce break apart synthetic molecules specifically designed to interact with specific enzymes. These molecules are sugars that are attached to fluorescent tags. There are a variety of reagents that will interact with each enzyme from the specific bacteria strains. Table 1 includes a list of reagents that can be used to react with each target enzyme.

TABLE 1

| Target Enzyme | Reagents |
|---|---|
| β-Galactosidase | Resorufin β-D-galactopyranoside (Res-Gal) |
| | 4-Methylumbelliferyl β-D-galactopyranoside (MUG) |
| | Fluorescein di-β-D-galactopyranoside (FDG) |
| | Carboxyumbelliferyl β-D-galactopyranoside (CUG) |
| | Indoxyl β-D-galactopyranoside |
| | 8-Hydroxyquinoline β-D-galactopyranoside |
| | 5-Bromo-4-chloro-3-indolyl β-D-galactopyranoside |
| | Phenyl-β-D-galactopyranoside |
| β-Glucuronidase | 4-Methylumbelliferyl β-D-glucuronide (MUGlcU) |
| | Carboxyumbelliferyl β-D-glucuronide (CUGlcU) |
| | Fluorescein di-β-D-glucuronide |
| | 4-Nitrophenyl β-D-glucuronide |
| | 5-Bromo-4-chloro-3-indolyl β-D-glucuronide |
| | Phenyl-β-D-glucuronide |
| | Resorufin β-D-glucuronide |
| | 6-Chloro-3-indolyl-β-D-glucuronide |
| | 8-Hydroxyquinoline-β-D-glucuronide |
| β-Glucosidase | Fluorescein di-β-D-glucopyranoside (FDGlu) |
| | Resorufin β-D-glucopyranoside |
| | 2-Nitrophenyl β-D-glucopyranoside |
| | 4-Methylumbelliferyl β-D-glucopyranoside |
| | 5-Bromo-4-chloro-3-indolyl β-D-glucopyranoside |
| | 6-Chloro-3-indolyl β-D-glucopyranoside |
| | carboxyumbelliferyl β-D-glucopyranoside |

When the enzymes react with these reagents, breaking the reagents apart, fluorescent tags, i.e., fluorophores, are produced with properties that can be detected in the sensor. More specifically, various embodiments employ a light source, such as a light emitting diode with a specific wavelength that excites a fluorophore near its maximum adsorption wavelength. Thereby, the fluorophore, if present, is prompted to re-emit energy, which can be detected by at least one optical detector.

The fluorescent tags produced by various reactions between a bacterially-produced enzyme and a reagent absorb light at certain wavelengths and then re-emit that absorbed light (emission) at a different wavelength. Table 2 details the properties of the different fluorescent compounds used by a sensor, according to one embodiment, in detection. More specifically, Table 2 shows the compounds detected in a multi-analyte sensor and their associated absorbance and emission wavelengths.

TABLE 2

| | Absorbance | Emission wavelength |
|---|---|---|
| Compound | wavelength (nm) | (nm) |
| Resorufin | 571 | 585 |
| 4-Methylumbelliferone | 360 | 499 |
| Fluorescein | 494 | 521 |
| Carboxyumbelliferone | 386 | 445 |
| Indoxyl | 280 | 398 |
| 8-Hydroxyquinoline | 255 | 325 |
| 5-Bromo-4-chloro-3-indole | 488 | 615 |
| Phenol | 260 | 290 |
| 4-Nitrophenol | 320 | 405 |
| 2-Nitrophenol | 337 | 415 |
| 6-Chloro-3-indole | 420 | 540 |

For example, Resorufin can be produced by a reaction between the reactant Resorufin β-D-galactopyranoside and the enzyme β-Galactosidase. The enzyme β-Galactosidase is indicative of the presence of coliforms, which are, in turn, indicative of fecal contamination. Various embodiments of the invention comprise mixing a water sample with the reactant, Resorufin β-D-galactopyranoside, to produce a mixed sample. If the water comprises the bacterial coliforms, which is indicative of fecal contamination, then the reactant, Resorufin β-D-galactopyranoside will react with the enzyme produced by the coliform bacteria, to produce Resorufin. Therefore, various embodiments of the invention further comprise irradiating that mixed sample with light having a wavelength from 500-800 nm, preferably from 550 to 700 nm, more preferably about 571 nm. As shown in Table 2, Resorufin has an absorbance wavelength of about 571 nm and, when exposed to light having that wavelength, Resorufin will emit light having a wavelength of 585 nm. The emitted light can be detected and it can be reliably determined that the water sample comprises the coliform bacteria, which is indicative of contamination with fecal matter.

4-Methylumbelliferone can be produced by a reaction between the reactant(s) 4-Methylumbelliferyl-β-D-galactopyranoside or 4-Methylumbelliferyl-β-D-glucuronide and the enzyme(s) galactopyronisidase or glucuronidase which are indicative of bacterial coliforms and E. coli respectively. Therefore, various embodiments of the invention comprise irradiating a sample comprising water and the reactant(s) 4-Methylumbelliferyl-β-D-galactopyranoside or 4-Methylumbelliferyl-β-D-glucuronide with light having a wavelength of from 300-400 nm, preferably from 320-380 nm, more preferably about 360 nm. If, in response to the irradiation, emitted light having a wavelength of about 499 nm is detected, it can be surmised that 4-Methylumbelliferone is present in the water sample, indicating contamination.

Fluorescein can be produced by a reaction between the reactant Fluorescein di-β-D-glucopyranoside and the enzyme(s) glucosidase, which is indicative of enterococci. Therefore, various embodiments of the invention comprise irradiating a sample comprising water and the reactant(s) Fluorescein di-β-D-glucopyranoside with light having a wavelength of from 400-550 nm, preferably from 450-500 nm, more preferably about 494 nm. If, in response to the irradiation, emitted light having a wavelength of about 521 nm is detected, it can be surmised that Fluorescein is present in the water sample, indicating contamination.

Carboxyumbelliferone can be produced by a reaction between the reactant(s) CUG, CUGlcU or carboxyumbelliferyl-β-D-glucopyranoside and the enzyme(s) galactosidase, glucuronidase, or glucosidase which are indicative of the bacteria E. coli, coliforms, or enterococci respectively. Therefore, various embodiments of the invention comprise irradiating a sample comprising water and the reactant(s) CUG, CUGlcU or carboxyumbelliferyl-β-D-glucopyranoside with light having a wavelength from 300 to 500 nm, preferably from 350 to 400 nm, more preferably about 386 nm. If, in response to the irradiation, emitted light having a wavelength of about 445 nm is detected, it can be surmised that Carboxyumbelliferone is present in the water sample, indicating contamination.

Indoxyl can be produced by a reaction between the reactant Indoxyl-β-D-glucuronide and the enzymeglucuronidase, which is indicative of the bacteria E. coli. Therefore, various embodiments of the invention comprise irradiating a sample comprising water and the reactant Indoxyl-β-D-glucuronide with light having a wavelength from 200 to 350 nm, preferably from 250 to 300 nm, more preferably about 280 nm. If, in response to the irradiation, emitted light having a wavelength of about 398 nm is detected, it can be surmised that Indoxyl is present in the water sample, indicating contamination.

8-Hydroxyquinoline can be produced by a reaction between the reactant(s) 8-Hydroxyquinoline-β-D-galactopyranoside and the enzyme(s) galactosidase, which is indicative of coliform bacteria. Therefore, various embodiments of the invention comprise irradiating a sample comprising water and the reactant(s) Hydroxyquinoline-β-D-galactopyranoside with light having a wavelength of from 200 to 300 nm, preferably from 225 to 275 nm, more preferably about 255 nm. If, in response to the irradiation, emitted light having a wavelength of about 325 nm is detected, it can be surmised that 8-Hydroxyquinoline is present in the water sample, indicating contamination.

5-Bromo-4-chloro-3-indole can be produced by a reaction between the reactant(s) 5-Bromo-4-chloro-3-indolyl-β-D-glucopyranoside and the enzyme(s) glucosidase which is indicative of the bacteria enterococci. Therefore, various embodiments of the invention comprise irradiating a sample comprising water and the reactant(s) 5-Bromo-4-chloro-3-indolyl-β-D-glucopyranoside with light having a wavelength of from 350 to 550 nm, preferably from 400 to 500 nm, more preferably about 488 nm. If, in response to the irradiation, emitted light having a wavelength of about 615 nm is detected, it can be surmised that 5-Bromo-4-chloro-3-indole is present in the water sample, indicating contamination.

Phenol can be produced by a reaction between the reactant(s) Phenyl-β-D-glucuronide and the enzyme(s) glucuronidase, which is indicative of the bacteria E. coli. Therefore, various embodiments of the invention comprise irradiating a sample comprising water and the reactant(s) Phenyl-β-D-glucuronide with light having a wavelength of from 200 to 320 nm, preferably from 230 to 290 nm, more preferably about 260 nm. If, in response to the irradiation, emitted light having a wavelength of about 290 nm is detected, it can be surmised that Phenol is present in the water sample, indicating contamination.

4-Nitrophenol can be produced by a reaction between the reactant(s) 4-Nitrophenyl-β-D-glucuronide and the enzyme(s) glucuronidase, which is indicative of the bacteria E. coli. Therefore, various embodiments of the invention comprise irradiating a sample comprising water and the reactant(s) 4-Nitrophenyl-β-D-glucuronide with light having a wavelength of from 250 to 400 nm, preferably from 300 to 350 nm, more preferably about 320 nm. If, in response to the irradiation, emitted light having a wavelength of about 405 nm is detected, it can be surmised that 4-Nitrophenol is present in the water sample, indicating contamination.

2-Nitrophenol can be produced by a reaction between the reactant(s) 2-Nitrophenyl-β-D-glucopyranoside and the enzyme(s) glucosidase, which is indicative of the bacteria enterococci. Therefore, various embodiments of the invention comprise irradiating a sample comprising water and the reactant(s) 2-Nitrophenyl-β-D-glucopyranoside with light having a wavelength of from 250 to 400 nm, preferably from 300 to 360 nm, more preferably about 337 nm. If, in response to the irradiation, emitted light having a wavelength of about 415 nm is detected, it can be surmised that 2-Nitrophenol is present in the water sample, indicating contamination.

6-Chloro-3-indole can be produced by a reaction between the reactant(s) 6-Chloro-3-indolyl-β-D-glucopyranoside and the enzyme(s) glucosidase which is indicative of the bacteria enterococci. Therefore, various embodiments of the invention comprise irradiating a sample containing water and the reactant(s) 6-Chloro-3-indolyl-β-D-glucopyranoside with light having a wavelength of from 350 to 500 nm, preferably from 400 to 450 nm, more preferably about 420 nm. If, in response to the irradiation, emitted light having a wavelength of about 540 nm is detected, it can be surmised that 6-Chloro-3-indole is present in the water sample, indicating contamination.

The devices and methods according to various embodiments can utilize each or a combination of any of these reagents without human interaction to detect the presence of bacteria indicating fecal contamination of a body of water.

### Apparatus

Various embodiments relate to an apparatus comprising a reaction chamber, a reversible pump, a reagent source, a light source, an optical detector, and a pre-programmed computer controller, which can comprise at least a processor and a memory. The reversible pump can have an inlet and an outlet. The outlet can be fluidically coupled to the reaction chamber. The inlet can be disposed to allow for collection of a water sample external to the reaction chamber.

The reagent source can comprise a fluorophore-tagged reagent. The reagent source can be fluidically coupled to the reaction chamber. The light source can emit light having a known wavelength. The light source can be positioned to expose at least a portion of the reaction chamber to the light having a known wavelength. The optical detector can be disposed to detect fluorescence emitted from the reaction chamber in response to light emitted from the light source.

The computer controller can be programmed to activate the reversible pump to deliver the water sample to the reaction chamber, to prompt delivery of the fluorophore-tagged reagent from the reagent source to the reaction chamber, to activate the light source to expose at least a portion of the reaction chamber to the light having a known wavelength, to receive a measurement of fluorescence intensity from the optical detector, to determine a concentration of at least one bacteria in the water sample based on the measurement of fluorescence intensity, and/or to report the concentration of the at least one bacteria in the water sample.

Multiple light sources would be used in the preferred embodiment. A white light source can be used to provide the entire spectrum of excitation lighting required, or a light source combined with the use of filters can be used for greater sensitivity of the detector. A mixture of reagents is designed to selectively indicate the presence of pathogenic contamination. The reagents can be added at from 50 to 500 μM, preferably from 100 to 300 μM, more preferably at about 150 μM for each bacteria type that is desired to be detected. One reagent can be selected from at least one of the categories in Table 1, but as many as three can be selected from Table 1, with no more than one from each category.

Reagent mixtures would preferably contain Carboxyumbelliferyl β-D-glucuronide, Fluorescein di-β-D-glucopyranoside, and Resorufin β-D-galactopyranoside to provide identification of E. coli, enterococci, and coliform bacteria selectively. When more than one compound from any category is chosen, they will each compete for the same enzymatic activity. In that case, the compound which is most easily reacted will be cleaved first, but other reactions will occur simultaneously. Detection is facilitated when at most one compound from each category in Table 1 is chosen.

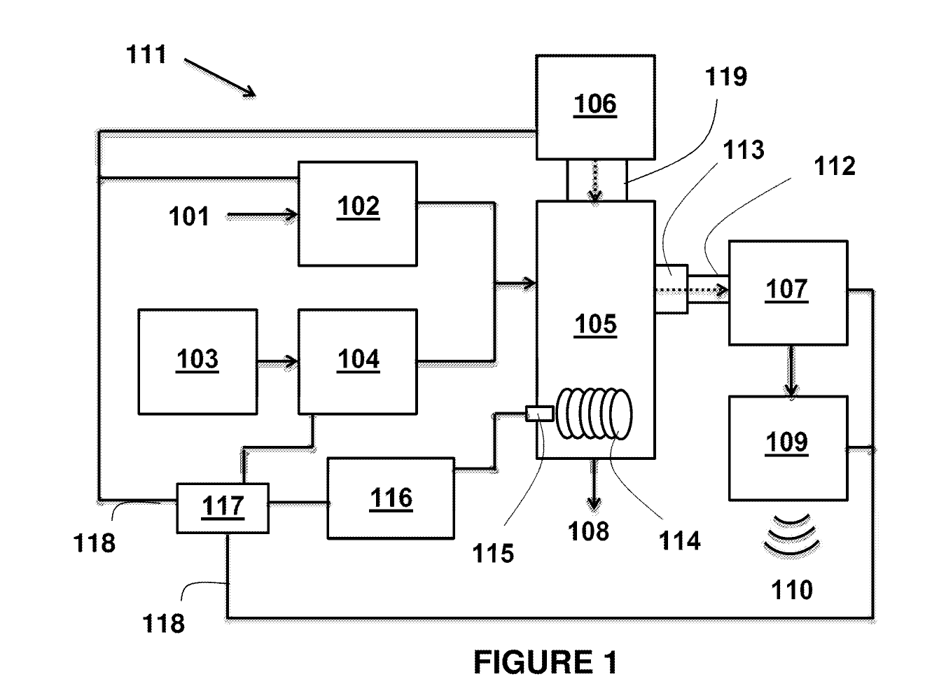

FIG. 1 provides a schematic block diagram of a system according to the present invention. As shown in FIG. 1, an inlet sample stream 101 can be collected from outside of the sensor system 111 and pumped into the system 111 by pump 102. More specifically, pump 102 can collect a sample external to the sensor system 111 and pump it into reactor 105. In reactor 105, the water sample from sample stream 101 and a reagent from reagent storage container 103 can be mixed and allowed to react over time. Reagent storage container 103 can contain chemicals utilized in reactor 105 to detect pathogens.

The chemicals used to detect pathogens can be pumped from reagent storage container 103 with pump 104 to reactor 105 to detect released optical tags. A light source or light sources 106 that targets the peak absorbance of the reagent optical tag(s) from reagent storage 103 can be employed along with a photodiode 107 or charged coupled device that collects the excited light emitted from the reagents. The light source(s) shines into the reactor through the quartz glass by a collimating lens 119 or by close proximity. The reactor must be sealed to exclude any outside light contamination. A sample discharge line 108 can also be provided from reactor 105 to flush used reactants out of the reactor 105. A wireless transmitter or a data logger 109 can be employed to report or record data from photodiode 107. A wireless signal 110 transmitted from wireless transmitter or a data logger 109 can be connected to a networked location. The entire sensor system 111 can be contained in a water proof case that encloses all of the components of the system.

Inside the sample chamber 105, the sample can be mixed with a specific aliquot of reagents. The mixture of reagents is designed to selectively indicate the presence of fecal contamination. One reagent can be selected from at least one of the categories in Table 1, but as many as three can be selected from Table 1, preferably with no more than one from each category. For each bacteria type that is desired to be detected, one or more reagents can be added in an amount within a range having a lower limit and/or an upper limit. The range can include or exclude the lower limit and/or the upper limit. The lower limit and/or upper limit can be selected from 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 μM. For example, according to certain preferred embodiments, for each bacteria type that is desired to be detected, one or more reagents can be added in an amount of about 150 μM.

Again, the configuration of the system can include an inlet pump 102 that collects external samples and pumps them into a custom designed stop-flow reactor system 105. The reactor system 105 contains at least one, but preferably three excitation light sources 106 that excite the hydrolyzed fluorophores within the reactor 105 and allow for optical detection through a fiber optic connection 112 coupled with collimating optical lenses 113. The custom designed reactor is configured with a resistive heating element 114 paired with a thermocouple 115 to a temperature controller 116 that can be dedicated or integrated into a microprocessor. The microprocessor or computer controller (as recited above) can be used to control the temperature controller 16, the light sources 106, the photodiode 107, the pumps 102, 104, the data-logger 109, and all other components of the system.

To approximate ideal physiological conditions where enzymes will have the most consistent and reproducible results, the reactor 105 can be maintained at a temperature within a range having a lower limit and/or an upper limit. The range can include or exclude the lower limit and/or the upper limit. The lower limit and/or upper limit can be selected from 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, and 130 degrees Fahrenheit. For example, according to certain preferred embodiments, to approximate ideal physiological conditions where enzymes will have the most consistent and reproducible results, the reactor 105 can be maintained at a temperature of from 80 to 120 degrees Fahrenheit, preferably from 90 to 100 degrees Fahrenheit, more preferably at about 98 degrees Fahrenheit with a tolerance of ±4 degrees Fahrenheit.

The fiber optic waveguide 112 can then transmits the collected light to a spectrometer 107 which processes the signal as absolute fluorescent intensity. After a natural water sample is pumped into the reactor chamber 105, a mixture containing multiple reagents and a buffer is pumped into the reactor system 105. For example, such a mixture can be comprised of Carboxyumbelliferyl-β-D-glucuronide, Fluorescein di-β-D-glucopyranoside, and Resorufin β-D-galactopyranoside to provide identification of E. coli, enterococci, and coliform bacteria selectively. This mixture can also contain nutrients to be utilized for the culturing of the bacteria present in the reactor.

The substrates (fluorophore tagged sugars are mixed with reverse osmosis filtered water and buffered with 100 mM phosphate buffer adjusted to a pH of 6.9, with an acceptable range from pH 6.0 to 7.5. Acceptable pH can be within a range having a lower limit and/or an upper limit. The range can include or exclude the lower limit and/or the upper limit. The lower limit and/or upper limit can be selected from 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, and 8. For example, according to certain preferred embodiments, acceptable pH can be within a range of from pH 6.0 to 7.5.

Rapid detection of the hydrolyzed compounds can be achieved with concentrations of each of the reagents within a range having a lower limit and/or an upper limit. The range can include or exclude the lower limit and/or the upper limit. The lower limit and/or upper limit can be selected from 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, and 360 μM. For example, according to certain preferred embodiments, rapid detection of the hydrolyzed compounds can be achieved with concentrations of each of the reagents in the range of 150 μM to 350 μM.

One mL of mixed reagent solution containing the tagged sugar complexes can be combined with 4 mL of the test-water sample inside of the custom reactor system to obtain the correct concentrations in order to facilitate rapid detection of the hydrolyzed compounds. Inside of the reactor, the target organism produces organism specific enzymes which will specifically cleave the fluorophore from the tagged sugar complex that corresponds to the organism specific enzyme

As shown in FIG. 1, a power source 117 can supply power to all components in need thereof, including but not limited to the pumps 102, 104, to the temperature controller 116, the thermocouple 115, to the light source(s) 106, to the photodiode 107, and to the data logger/transmitter 109. A variety of power sources are available. The power can be supplied via power lines 118. The inclusion of 12 V batteries provides the electricity required to power the pumps and sensors in the device. Another option is the use of solar collectors to recharge the batteries. Through the capturing of solar energy on photovoltaic cells, the sensor could be run indefinitely. The use of a submerged turbine could also function as a power source in moving waters (provided the sensor is anchored in place). A combination of these power sources could allow the sensor to continuously monitor water quality in a wide range of environmental conditions.

Upon hydrolysis through enzymes produced by fecal coliforms, such as, enterococci, and Escherichia coli; these reagents from Table 1 can release glycosidically-bound fluorophores from the reagents into solution. The fluorophores are excited by light emitting diodes at different wavelengths selected upon the absorbance wavelength of the released fluorophore that results in the optimal excitation of each released fluorophore. A charge coupled device array sensor records the change in fluorescent intensity at the peak emission wavelength, and values are wirelessly transmitted to an external, networked location. Alternatively, these values may be collected internally with a data logger. One sample is analyzed over an extended period of time so that fluorescent intensity data as a function of time can be collected.

The algorithm to correlate fluorescent intensity to bacteria concentration is calculated by relating the time the ratio of current fluorescent intensity to initial fluorescent intensity to exceed a value of 1.1. Upon reaching a threshold predetermined by a confidence interval of from 50 to 100%, preferably from 60 to 90%, more preferably by a confidence interval of at least an 85%, a calculation of the number of bacteria in a 100 mL sample can be established. The more rapid the threshold time is achieved the higher the number of CFU/100 mL is calculated and quantified for an individual sample. As hydrolysis is a kinetically limited process, the initial reading of the sensor's fluorescence intensity is not influenced significantly by the initial hydrolysis that occurs when the external sample mixes with the internal reagent mixture and can be used as a background for each sample. Stability of the reagents in the natural environment decreases and establishing a new background for each sample helps eliminate any potential issues with reagent degradation.

For sensors in which the local data logging occurs, there can be a regular collection of data in a memory for analysis. In systems with a wireless or wired network, the data can be collected and available for real-time analysis. In such case, there will still be the need for an operator to attend the sensor and periodically remove the spent reagents which have been held in the collection chamber. This will be done to ensure that there is no reagent released into the environment which could affect the readings of nearby sensors.

While the direct measurement of the number of bacteria present per sample is costly and time-intensive, the device according to various embodiments will serve to use the indirect measurement of enzyme activity through the released fluorophore moiety of saccharide-based reagents to correlate luminescence or fluorescence to bacteria concentrations. Through proprietary correlations, pathogen concentrations in the water sample are determined and used to report instances when counts have exceeded acceptable thresholds. As a result, water quality officials can make decisions quicker and respond to pathogenic events more accurately and rapidly. Sensors, according to various embodiments, can detect bacteria using the enzymatically catalyzed glycosidases. E. coli, for example, secrete β-D-glucuronidase into their environment. When the compound carboxyumbelliferyl β-D-glucurconide is introduced into a buffered solution containing β-D-glucuronidase, this enzyme hydrolyzes the bond between the glucuronicsaccharide and the carboxyumbelliferone moiety (a fluorophore), releasing and activating it. This freed fluorophore absorbs UV light and emits light at a visible wavelength, while the enzyme is available to again catalyze this same reaction. Sensors, according to various embodiments, can utilize this and similar enzymatic reactions to determine the presence of pathogens in a body of water.

As fluorescence increases faster with greater concentrations of bacteria (and thus of extracellular enzyme), the timed rate of increase for fluorescence is used to correlate direct sensor measurements with the presence of bacteria. The sensors, according to various embodiments can work in a networked grid with multiple other identical units. As water conditions change rapidly and contamination moves, signals indicating the presence of fecal coliform bacteria indicate the magnitude and severity of incidents such as CSO discharges. Correlations developed allow not only the tracking of contamination in a body of water across a sensor grid, but also the prediction of future advances of the contamination.

The technology can take the form of an entirely hardware embodiment, or an embodiment containing both hardware and software elements. In one embodiment, the invention is implemented in software, which includes but is not limited to firmware, resident software, microcode, etc. Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium (though propagation mediums in and of themselves as signal carriers are not included in the definition of physical computer-readable medium). Examples of a physical computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-RAN) and DVD. Both processors and program code for implementing each as aspect of the technology can be centralized and/or distributed as known to those skilled in the art.

### Methods

Various embodiments relate to methods comprising adding a tagged reagent to a water sample, wherein the reagent is water soluble; and determining a concentration of at least one bacteria in the water sample based on an intensity of an emission emitted from the water sample in response to exposure to light having a known wavelength. The tagged reagent is a fluorophore-tagged reagent and the intensity is a fluorescence intensity. The tagged reagent is a colorimetric-tagged reagent and the intensity is an emission light intensity.

Various embodiments relate to methods comprising adding a fluorophore-tagged reagent to a water sample. The fluorophore-tagged reagent can be water soluble and/or hydrophilic. The water sample can be of a type selected from the group consisting of saline water, brackish water, fresh water, and combinations thereof. The fluorophore-tagged reagent can comprise a sugar. The fluorophore-tagged reagent can be hydrophilic and water soluble. A fluorophore can be cleaved from the fluorophore-tagged reagent in the presence of an enzyme selected from the group consisting of β-D-galactopyranosidease, β-D-glucopyranosidase, β-D-glucuronidase, and combinations thereof. A fluorophore can be cleaved from the fluorophore-tagged reagent in the presence of at least one type of bacteria, and the light having a known wavelength can excite the fluorophore near its maximum adsorption wavelength.

The method can further comprise determining a concentration of at least one bacteria in the water sample based on a fluorescence intensity emitted from the water sample in response to exposure to light having a known wavelength. The known wavelength can be selected from the group consisting of an ultraviolent wavelength, a visible wavelength, an infrared wavelength, a near infrared wavelength, and combinations thereof. The step of determining a concentration of at least one bacteria in the water sample based on a fluorescence intensity emitted from the water sample in response to exposure to light having a known wavelength can be completed within a time within a range having a lower limit and/or an upper limit. The range can include or exclude the lower limit and/or the upper limit. The lower limit and/or upper limit can be selected from 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, 20, 20.2, 20.4, 20.6, 20.8, 21, 21.2, 21.4, 21.6, 21.8, 22, 22.2, 22.4, 22.6, 22.8, 23, 23.2, 23.4, 23.6, 23.8, 24, 24.2, 24.4, 24.6, 24.8, 25, 25.2, 25.4, 25.6, 25.8, 26, 26.2, 26.4, 26.6, 26.8, 27, 27.2, 27.4, 27.6, 27.8, 28, 28.2, 28.4, 28.6, 28.8, 29, 29.2, 29.4, 29.6, 29.8, and 30 hours. For example, according to certain preferred embodiments, the step of determining a concentration of at least one bacteria in the water sample based on a fluorescence intensity emitted from the water sample in response to exposure to light having a known wavelength can be completed within a time selected from 1 to 24 hours. Similarly, the determining step can take less than 24 hours, from 1 to 24 hours, from 5 to 20 hours, or from 10 to 15 hours.

The method can further comprise measuring a threshold time that is required for the fluorescence intensity emitted from the water sample to reach a predetermined threshold, and the step of determining the concentration of the at least one bacteria in the water sample is also based on the threshold time. The method can further comprise reporting the concentration of at least one bacteria in the water sample. The reporting step can comprise wirelessly transmitting data indicating the concentration of at least one bacteria to one selected from the group consisting of at least one networked location, at least one data logger, and combinations thereof. The method can further comprise emitting the light having a known wavelength from a light emitting diode. The method can further comprise detecting the fluorescence intensity with a photodiode. The method can further comprise determining a concentration of a plurality of strains of bacteria in the water sample. The method can further comprise maintaining the water sample at a temperature within a range having a lower limit and/or an upper limit. The range can include or exclude the lower limit and/or the upper limit. The lower limit and/or upper limit can be selected from 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, and 130 degrees Fahrenheit. For example, according to certain preferred embodiments, the method can further comprise maintaining the water sample at a temperature of from about 90 to 120 degrees Fahrenheit.

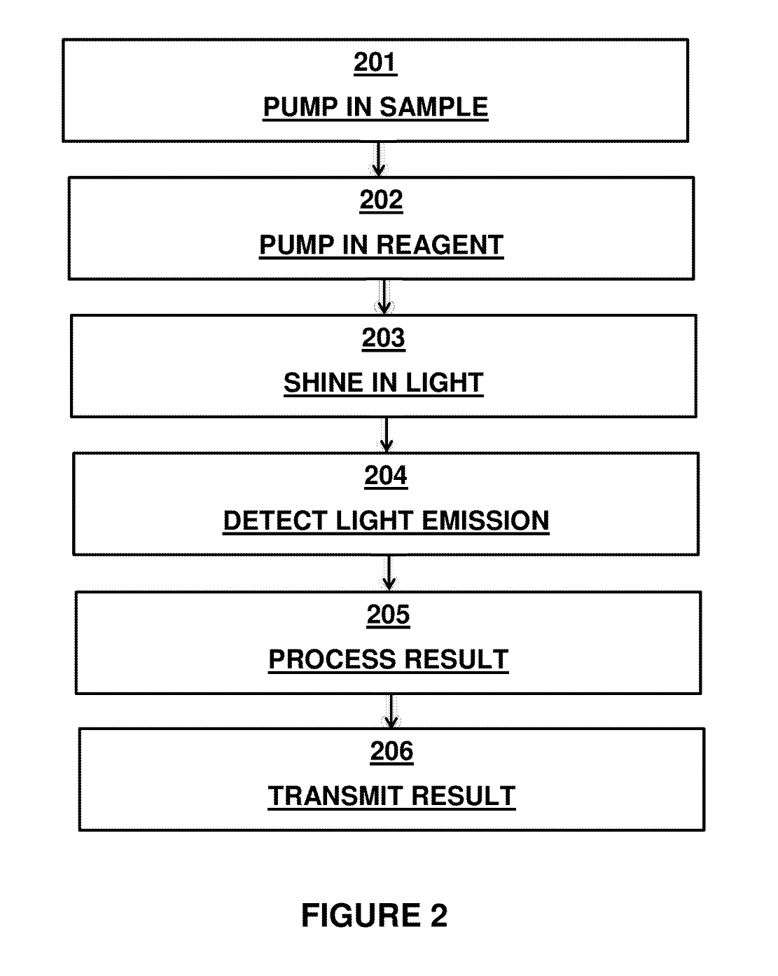

According to various embodiments, a sensor can be enclosed in a waterproof case securing all internal components. FIG. 2 is a functional block diagram, illustrating steps of operating a device according to various embodiments. Water samples are automatically drawn at timed intervals to a sample chamber via tubing that collects a sample external to the sensor. As shown at box 201, the sample is pumped into the reaction chamber where the detection occurs. Next, at box 202 a reagent is pumped into the reaction chamber. At box 203, a light is emitted into the reaction chamber from one or more light sources 106 that target the peak absorbance of the reagent's optical tag(s). At box 204, a photodiode or a charged coupled device detects excited light emitted from the reagents in the reaction chamber. At box 205, the results are processed, and at box 206 the results are transmitted. Upon completion of monitoring a specific sample, the spent solution is pumped into a collection chamber and the reactor chamber is cleaned either with a rinsing solution or a natural sample collected externally to purge the chamber by creating a turbulent environment that detaches immobilized cells. After this washing, the sensor repeats measurements as dictated by an automated schedule or microprocessor that starts a new cycle once a threshold is achieved or not achieved during the specified time period.

Various embodiments relate to a method of simultaneously detecting a concentration of each of a plurality of target organisms in a water sample. The method can include adding one or more tagged reagents to the water sample comprising a plurality of target organisms; exposing the water sample to light having a known wavelength; detecting a plurality of light emissions from the water sample; and determining, by a processor, the type and the concentration of each of the plurality of target organisms in the water sample by detecting an intensity for each of the plurality of light emissions from the water sample. The determining step can include distinguishing, by a processor, between the one or more tagged reagents by comparing a plurality of emission peaks.

Each of the plurality of target organisms can produce a species-specific byproduct, and each of the one or more tagged reagents can interact with one of the species-specific byproducts to emit a unique light emission from the water sample. The water sample can be from a natural environment, and the natural ranges of turbidity occurring in the water sample do not impact the ability of the sensor to detect optical signatures of the species-specific by-products interactions with the one or more tagged reagents. Each of the plurality of target organisms can be selected from E. coli, coliforms, and Enterococcus.

The one or more tagged reagents can be water soluble and flowable in a liquid medium such that they can be pumped from one area to another, for example, within an apparatus according to various other embodiments. The one or more tagged reagents can be selected such that they do not impact the ability of the plurality of target organisms to continue to grow after sampling. The one or more tagged reagents can be or can include a food source for the target organisms.

The one or more tagged reagents can have a peak emission separation within a range having a lower limit and/or an upper limit. The range can include or exclude the lower limit and/or the upper limit. The lower limit and/or upper limit can be selected from 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, and 1500 nm. For example, according to certain preferred embodiments, the one or more tagged reagents can have a peak emission separation of at least 50 nm.

The one or more tagged reagents can be distinguishably detected at a peak emission separation within a range having a lower limit and/or an upper limit. The range can include or exclude the lower limit and/or the upper limit. The lower limit and/or upper limit can be selected from 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, and 1000 nm. For example, according to certain preferred embodiments, the one or more tagged reagents can be distinguishably detected at a peak emission separation of from 100-1000 nm.

Each light emission of the plurality of light emissions can change over time and the change of each light emission of the plurality of light emissions over time can corresponds to an initial concentration of one of the plurality of target organisms present in the water sample.

Each light emission of the plurality of light emissions can change over time, and the change in one of the plurality of light emissions over a time period of less than 6 hours can correspond to an amount of pre-existing by-products of at least one of the plurality of target organisms in the water sample prior to addition of the one or more tagged reagents. Each light emission of the plurality of light emissions can change over time, and the change in one of the plurality of light emissions over a time period of more than 6 hours corresponds to an amount of by-products exudated by at least one of the plurality of target organisms after addition of the one or more tagged reagents. Each light emission of the plurality of light emissions can change over time, and an initial rate of change of the plurality of light emissions can correspond to a concentration of by-products of the plurality of target organisms that are present in the water sample. The by-products can be selected from the group consisting of metabolic materials and enzymatic materials.

The water sample can include by-products of the plurality of target organisms. Again, the by-products can be selected from the group consisting of metabolic materials and enzymatic materials, and the concentration of by-products in the water sample can correspond to the concentration of the plurality of target organisms in the water sample.

According to various embodiments, determining the type and the concentration of each of the plurality of target organisms in the water sample is not dependent on growth of the organisms.

The invention is further described in the following illustrative examples in which all parts and percentages are by weight unless otherwise indicated.

### EXAMPLES

### Materials

Three distinct fluorophores, Resorufin, Fluorescein, and 7-Hydroxycoumarin-3-Carboxylic acid [carboxylumbelliferone or HCC] can be identified by exciting them with a broad electromagnetic (EM) wave spectrum and their fluorescent emission can be detected with a photodetector. Each fluorophore has a distinct absorption peak and emission peak that is independent from the other fluorophores chosen. HCC is excited at ˜350 nm and emits at ˜447 nm. HCC gives a low intensity fluorescent signal compared to the other fluorophores. Fluorescein is excited at 495 nm and emits at 512 nm. Fluorescein emits a high intensity fluorescent signal compared to other fluorophores. Resorufin is excited at 570 nm and emits at 585 nm. Resorufin gives an average intensity fluorescent signal compared to other fluorophores. The incident EM wave spectrum to be used is between 350 nm and 750 nm.

### Instrumentation

A photo detector is used that has a collimator to insure the incident light travels through the sample in a straight parallel line. A fiberoptic cable is attached at a 90 degree angle to the incident light in order to transmit fluorescent scatter from the excited fluorophores without receiving any of the incident light. The geometry of the photodetector prevents the incident light from being detected by the photodetector. A super white LED (SWLED) light was used to introduce a broad range of EM wave spectrum to the sample. A Ultra-Violet LED (UV LED) was used to introduce an ultraviolet spectrum. The SWLED and UV LED were geometrically configured 180 degrees from one another with their bulbs facing each other so that the incident light provided by each LED would be parallel to one another and perpendicular to the detecting fiber optic and used an interchangeable resistor used to prevent a voltage or current that exceeds the LED's max voltage and current specifications.

### Example 1

The purpose of this example was to test a variety of monofluorophores. More specifically, Fluorescein, Resorufin, HCC with 50 msec integration time. The integration time is the amount of time incident light on the photodiode is allowed to collect and build up a signal before the photodiode is reset and then allowed to start a new integration time period.

Using a photo detector and LEDs as described above, varying concentrations of fluorophores were mixed with deionized water and placed into the instrument. The respective intensity was collected and is reported.

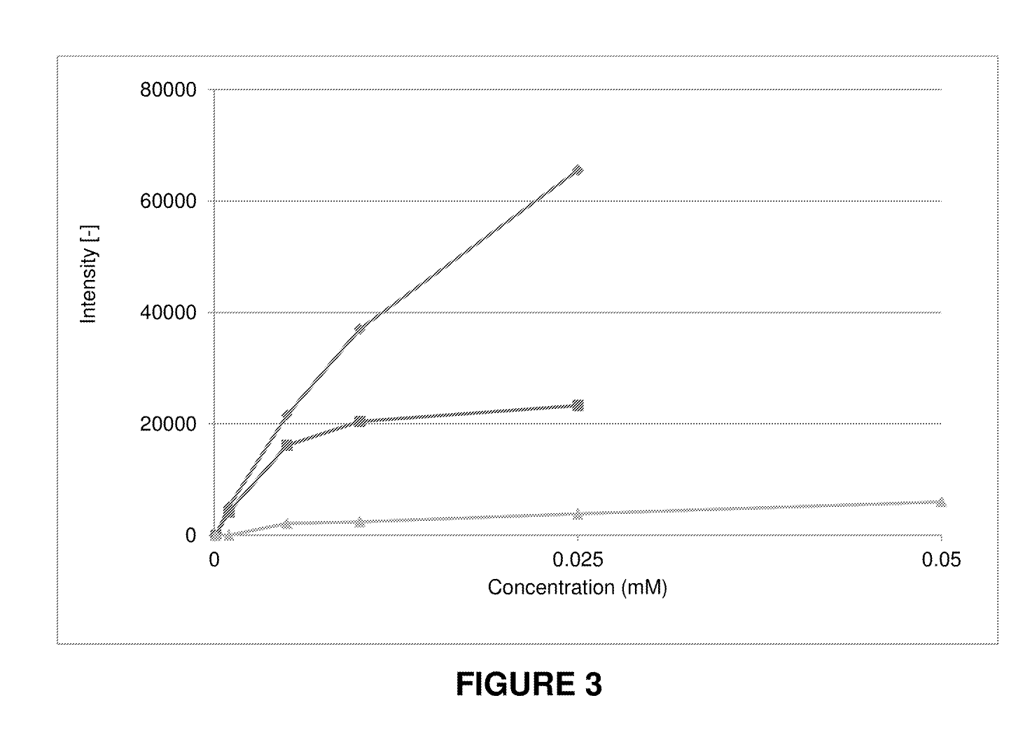

FIG. 3 is a chart showing different intensity responses to various concentrations of all three monofluorophores with the same integration time (50 msec). The photodetector becomes flooded at integration times higher then 50 msec for Fluorescein. ♦ represents fluorescein, ▴ represents HCC, and ▪ is resorufin.

Fluorescein fluorescent emission was very high compared to the other fluorophores and requires a short integration time of 30 msec. Resorufin fluorescent emission is not as intense as fluorescein and needed a longer integration time of 170 msec. HCC response to the excitation incident light is weaker and requires a longer integration time of 325 msec.

### Example 2

The purpose of this example was to test difluorophores. Using a photo detector and LEDs as described above, varying concentrations of fluorophores were mixed with each other, two at a time, and mixed with deionized water and placed into the instrument. The respective intensity was collected and is reported.

According to this example, two fluorophores, resorufin and fluorescein, were mixed with each other in the same assay at varying concentrations to determine the ability to separate and influence a photodetector sensor system. Table 3 summarizes information from the photospectrometer for the 25 mixtures made with fluorescein and Resorufin at varying concentrations. λ=520 nm corresponds with Fluorescein and λ=590 nm corresponds to Resorufin. Integration time of 40 msec.

TABLE 3

| | R: ↓ | | λ = 517 nm | | | | | λ = 582 nm | | | | |
|---|---|---|---|---|---|---|---|---|---|---|---|---|
| F: → | (mM) | | 0.025 | 0.01 | 0.005 | 0.0025 | 0.001 | 0.025 | 0.01 | 0.005 | 0.0025 | 0.001 |
| | | | ↓ | Intensity | | | ↓ | ↓ | Intensity | | | ↓ |
| | 0.025 | → | 34027 | 26085 | 17225 | 8206 | 4317 | 19850 | 17443 | 16673 | 15223 | 15739 |
| | 0.01 | Intensity | 41533 | 28510 | 17200 | 7833 | 4135 | 20517 | 18745 | 16469 | 15444 | 15569 |
| | 0.005 | | 45072 | 30726 | 15087 | 8831 | 4638 | 18990 | 18745 | 13467 | 15259 | 13644 |
| | 0.0025 | | 59724 | 30257 | 18945 | 9619 | 5175 | 17060 | 12697 | 10841 | 10970 | 8853 |
| | 0.001 | → | 65256 | 33587 | 17786 | 9748 | 5310 | 12195 | 9030 | 6470 | 6244 | 5826 |

Table 4 shows 517 nm fluorescent intensity range for set Fluorescein concentrations as Resorufin concentration change. As Fluorescein concentration decreases, the change in its peak range decreases as Resorufin changes.

TABLE 4
λ = 517 nm

| | Fluorescein (mM) | | | | |
|---|---|---|---|---|---|
| | 0.025 | 0.01 | 0.005 | 0.0025 | 0.001 |
| Peak intensity | 65256 to | 33587 to | 18945 to | 9748 to | 5310 to |
| range | 34027 | 26085 | 15087 | 7833 | 4135 |

Table 5 shows 582 nm Resorufin intensity range for set Resorufin concentrations as Fluorescein concentration change. Almost all concentration ranges overlap.

TABLE 5
λ = 582 nm

| | Resorufin (mM) | | | | |
|---|---|---|---|---|---|
| | 0.025 | 0.01 | 0.005 | 0.0025 | 0.001 |
| Peak | 19850 to | 20517 to | 18990 to | 17060 to | 12195 to |
| intensity | 15223 | 15444 | 13767 | 8853 | 5826 |
| range | | | | | |

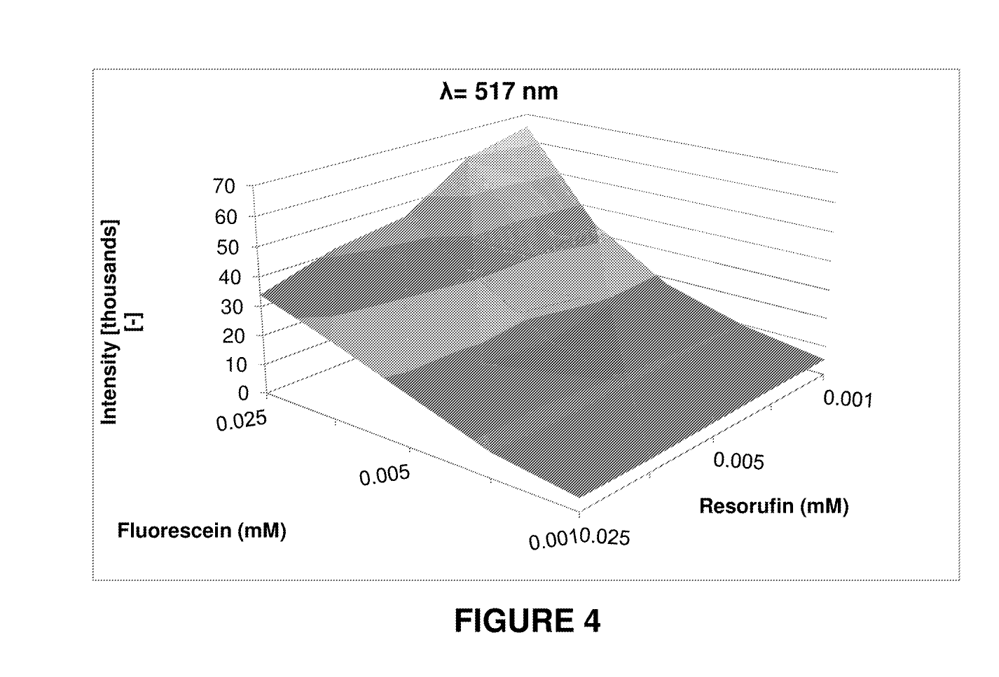

FIG. 4 is a chart displaying the recorded intensity at 517 nm as a function of fluorescein and resorufin concentration. The resorufins concentration decreases light intensity increases for a fixed concentration of fluorescein. For a fixed value of resorufin, there is a decrease in intensity as fluorescein concentration decreases. This is to be expected as λ at 517 nm is indicative of fluorescein.

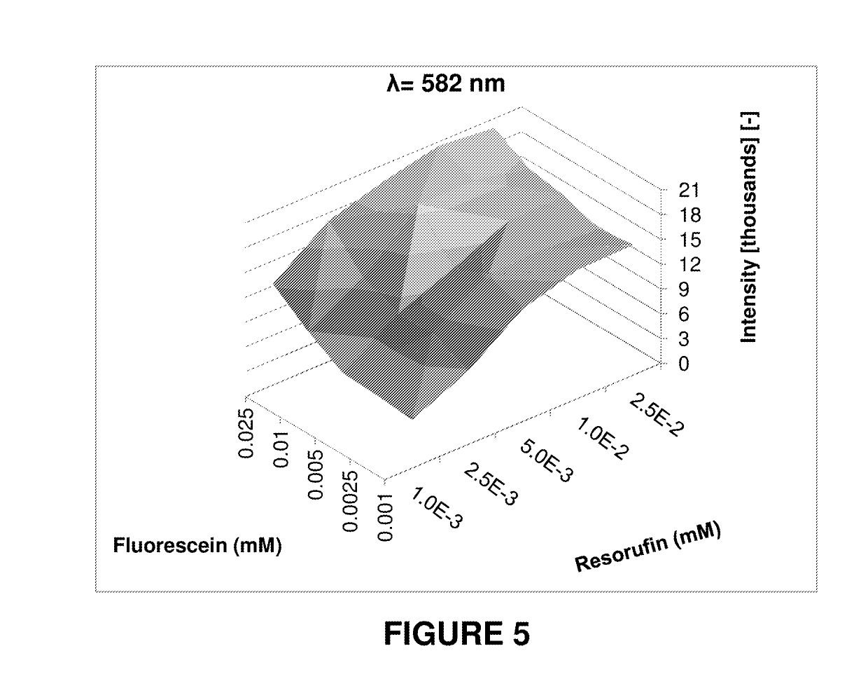

FIG. 5 is a chart showing the recorded intensity at 582 nm as a function of fluorescein and resorufin concentration. As resorufins concentration increases light intensity increases for a fixed concentration of fluorescein. For a fixed value of resorufin, there is a also an increase in intensity as fluorescein concentration increases. This must be accounted for in the optical sensor that the baseline threshold will increase during the assay when these two compounds are being detected in the sensor.

Fluorescein has a characteristic response at 517 nm and Resorufin has a characteristic response at 582 nm. At 512 nm there is an increase in light intensity for a fixed concentration of Fluorescein as Resorufin decreases. In spite of variations in the Fluorescein intensity peak levels, there is no major overlap between Fluorescein concentration level and peak intensity ranges with the exception of higher concentration levels of Fluorescein that are unlikely to be found in an optical sensor. Concentrations of Fluorescein under 0.01 mM will be easily distinguishable within a certain margin of error.

As concentrations of resorufin increase, fluorescent emission intensity increases. An important observation to notice is that as the Fluorescein concentration level increase and the concentration of Resorufin is fixed, fluorescent intensity increases. It is possible that Resorufin has an increased ability of being excited as the fluorescent intensity of Fluorescein is increases at 517 nm.

It is possible to detect the presence of Fluorescein and Resorufin in a sample. However, they do have an interaction with each other that affect their fluorescent intensity at certain concentrations. A mathematically model corrects for these variations so that an accurate concentration of each fluorophore can be determined from the intensity reading of their characteristic emission wavelengths. The two compounds can be utilized simultaneously in a sensor to produce a resulting spectrum of each compound that correlates with the bacteria concentration of its respective species it is being used to detect.

### Example 3

The purpose of this example was to test trifluorphores. Using a photo detector and LEDs as described above, varying concentrations of fluorophores were mixed with each other, then mixed with deionized water and placed into the instrument. The respective intensity was collected and is reported. Table 6 summarizes output intensity response for characteristic wavelengths. Each fluorophore was prepared and tested with one high level and two low levels. The fluorescent intensity and the rate of intensity growth correlate directly with the bacteria concentration inside of the reactor sensor assay.

TABLE 6

| | | | | Integration time 45 msec | | |
|---|---|---|---|---|---|---|
| | Trifluorophore | | | λ = | | |
| Sample | F, R, and HCC (mM) | | | 450 nm or | | |
| # | F: | R: | H: | 480 nm | λ = 518 nm | λ = 588 nm |
| 1 | 0.025 | 0.005 | 0.005 | 0 | 63221 | 27013 |
| 2 | 0.005 | 0.025 | 0.005 | 0 | 15548 | 28932 |
| 3 | 0.005 | 0.005 | 0.025 | 0 | 18653 | 18773 |

Each of the fluorophores, Fluorescein, Resorufin, and HCC, have a distinct wavelength from one another as well as differing fluorescent emission peak intensities. The most intense fluorescent emission intensities were observed from Fluorescein. The least intense fluorescent emission intensities were observed from HCC. Resorufin gave fluorescent emission intensities in between those observed from Fluorescein and HCC. Shorter integration times are needed for fluorophores with high emission intensities and longer integration times are needed for fluorophores with smaller emission intensities. When doing single species bacteria detection using fluorophores, the fluorophores are easily detectable if the integration time is adjusted to accommodate for the specific fluorophores emission intensity. However, when simultaneous detection of the fluorophores (i.e. multiple bacteria species) is required it becomes harder to identify HCC due to the emissivities and detection requirements of multiple bacteria species.

It is possible to independently detect the presence of a fluorescent agent whether that agent be part of a monofluorophore, difluorophore, or trifluorophore sample. The ability to easily detect the presence and concentration of a given fluorophore with photospectrometry is greatly increased when the fluorophore is being tested in a monofluorophore sample as integration time can be tailored for the specific fluorophore. As fluorophore samples become more complex, or heterogeneous, it becomes more difficult to determine the specific concentration of each fluorophore. Even though the determination of each fluorophore concentration in a di- or trifluorophore sample is more difficult than a monofluorophore sample, there is an intensity peaks that correlate with characteristic wavelengths for each fluorophore, indicating that the detection of the presence of each fluorophore can be easily determined. In order to determine the exact concentrations of fluorophores in di- and trifluorophore concentration, interactions between multiple fluorophores must be incorporated to the threshold algorithm to prevent over/under estimation of the bacteria concentration.

### Example 4

The purpose of this example is to demonstrate sensor construction and calibration. For sensor construction, anti-microbial tubing and connectors were purchased from Cole Palmer. The reactor is a custom milled aluminum alloy fitted with a 1 cm inner diameter quartz cell capable of holding 5 ml of liquid. A Watlow heater controller and Vulcan cartridge heater connected through a Watlow solid-state relay are used to heat the aluminum block to 44° C. to selectively grow enteric organisms (EPA 1986). Pumps are micro-diaphragm liquid pumps purchased from KNF Neuberger, Inc. Solenoid valves were purchased from Gems Sensors and Controls. The relay control board was purchased from National Control Devices, LLC. The charge coupled device (CCD) array spectrometer and light emitting diode (380 nm bulb) were purchased from Ocean Optics and configured for fluorescence analysis. Optical cables along with connectors were also purchased from Ocean Optics. Microsoft Visual Basic 6.0 Professional was used to write the controlling software and interface with the spectrometer. A waterproof Pelican™ case was used to protect the components from water and dust.

Carboxyumbelliferyl β-D-glucuronide was used in the development of a correlation curve (Marker Gene Tech, Eugene, Oreg.). The fluorescent substrate is mixed with Milli-Q water and buffered with 100 mM phosphate buffer (Sigma-Aldrich) adjusted to a pH of 6.9. Various concentrations of substrate were tested and it was determined a concentration of approximately 250 μM provides a sufficient fluorescent signal without excessive use of reagent (Geary, 2009). One mL of reagent mixture containing the fluorescent substrate is added to each 4 mL sample.

To develop a quantification curve, varying wild-strain E. coli concentrations were used to correlate increases in sample fluorescence to E. coli concentration. This curve facilitated the use of an algorithm that determines the time specific concentrations of bacteria need to reach an intensity threshold relative to initial fluorescent conditions. Intensity data was transmitted from the prototype every two minutes. When the ratio between initial fluorescence intensity and current intensity exceeded 1.1, we determined E. coli to be present above the EPA recreational water limits of 125 colony forming units (CFU)/100 mL (EPA 1986). In laboratory test, an E. coli concentration of 125 CFU/100 mL required 6.05 hrs with a standard error of 9.1 min (N=6) to reach the 1.1 threshold. For the demonstration we determined that if the 1.1 threshold is not reached in 8 hours the E. coli concentration is below the designated maximum concentration (determined by a 95% confidence interval for measurements at 125 CFU/100 mL) (Geary, 2009).

E. coli concentrations in all experiments were identified as the geometric mean of triplicate analysis by selective culturing media conducted using Coliscan Easygel (Microbiology Labs, Goshen, Ind.). Samples processed by the sensor were analyzed by selective media culturing prior to and after each run to ensure bacterial concentrations remained unchanged during the length of the assay. No statistically significant concentration changes were noticed during the assays.

Sample waters used throughout the experiments were diluted influent collected from the South Bend Waste Water Treatment Plant (SBWWTP), South Bend, Ind. Prior to and after sample introduction, the reactor and tubing were flushed for 30 seconds with sample water to purge any remaining fluorophore. After allowing the flushed water to drain, a 4 mL sample is introduced and retained in the reactor for analysis.

Utilizing the algorithm, a simulated combined sewage outfall event was introduced over a period of 16 hours to determine the viability of the system in alerting authorities to such an event. Samples were diluted to a 1:4 water/wastewater ratio for simulated combined sewage outfall water. Reproducibility test were conducted where samples were maintained on ice during three repeated assays to ensure constant bacteria concentrations. E. coli concentrations were measured at the beginning and end of the three repeated assays and showed minimal changes during the length of the assays.

Prior to sensor deployment, controlled sensor runs were conducted to establish a quantification curve. Concentrations of E. coli ranging from 68 CFU to 3000 CFU/100 mL were measured as a function of time required to reach the algorithm threshold. Times of detection (i.e. time to reach the 1.1 threshold at λ=365 nm) for EPA recreational water E. coli levels were found in the time range from 5-8 hrs while higher concentrations (>5000 CFU/100 mL), as would be found in a combined sewage outfall event, were detected in less than an hour. For example, samples of 2555 CFU/100 mL were assayed four times and had an average time of detection of 48 min and a standard deviation (STD) 17.22 min.

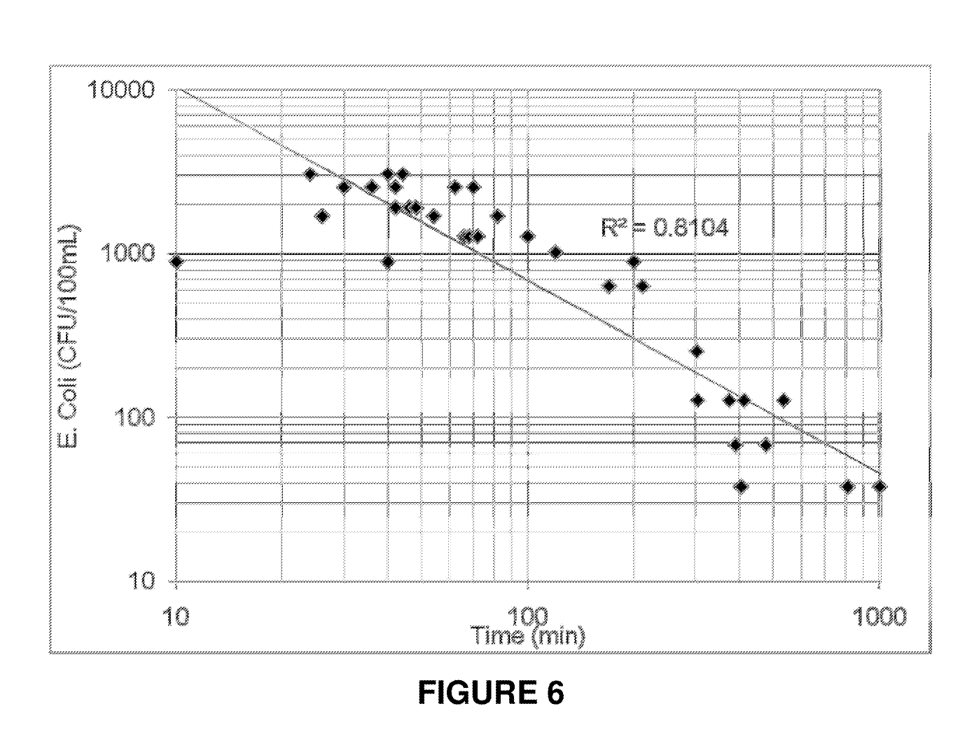

FIG. 6 shows a correlation curve showing that the time to reach the detection threshold varies with the concentration of the bacteria present in the sample. As shown in FIG. 6, the times to detection fit an exponential model with a linear regression coefficient (R2) of 0.8104 (n=35).

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C §112, sixth paragraph. In particular, the use of “step of” in the claims herein is not intended to invoke the provisions of 35 U.S.C §112, sixth paragraph.

What is claimed is:

1. A method comprising
adding a tagged reagent to a water sample, wherein the tagged reagent is water soluble; and
determining a concentration of at least one bacteria in the water sample based on an intensity of an emission emitted from the water sample in response to exposure to light having a known wavelength.

2. The method of claim 1, wherein the tagged reagent is a fluorophore-tagged reagent and the intensity is a fluorescence or colorimetric intensity.

3. The method of claim 1, wherein the tagged reagent is a colorimetric-tagged reagent and the intensity is an emission light intensity.

4. The method of claim 1, further comprising reporting the concentration of at least one bacteria in the water sample.

5. The method of claim 4, wherein the reporting step comprises wirelessly transmitting data indicating the concentration of at least one bacteria to one selected from the group consisting of at least one networked location, at least one data logger, and combinations thereof.

6. The method of claim 1, further comprising emitting the light having a known wavelength from a light emitting diode.

7. The method of claim 1, wherein a fluorophore is cleaved from the fluorophore-tagged reagent in the presence of at least one type of bacteria, and wherein the light having a known wavelength excites the fluorophore near its maximum adsorption wavelength.

8. The method of claim 1, further comprising detecting the fluorescence intensity with a photodiode.

9. The method of claim 1, comprising determining a concentration of a plurality of strains of bacteria in the water sample.

10. The method of claim 1, wherein the water sample is of a type selected from the group consisting of saline water, brackish water, fresh water, marine water, and combinations thereof.

11. The method of claim 1, wherein the determining step takes less than 24 hours.

12. The method of claim 1, wherein a fluorophore is cleaved from the fluorophore-tagged reagent in the presence of an enzyme selected from the group consisting of β-D-galactopyranosidease, β-D-glucopyranosidase, β-D-glucuronidase, and combinations thereof.

13. The method of claim 1, wherein the fluorophore-tagged reagent comprises a sugar.

14. The method of claim 1, wherein the fluorophore-tagged reagent is hydrophilic.

15. The method of claim 1, wherein the known wavelength is selected from the group consisting of an ultraviolent wavelength, a visible wavelength, an infrared wavelength, a near infrared wavelength, and combinations thereof.

16. The method of claim 1, further comprising maintaining the water sample at from about 90° F. to 120° F.

17. The method of claim 1, further comprising measuring a threshold time that is required for the fluorescence intensity emitted from the water sample to reach a predetermined threshold, and
wherein the step of determining the concentration of the at least one bacteria in the water sample is also based on the threshold time.

18. An apparatus comprising
a reaction chamber;
a reversible pump having an inlet and an outlet,
wherein the outlet is fluidically coupled to the reaction chamber,
wherein the inlet is disposed to allow for collection of a water sample external to the reaction chamber;
a reagent source comprising a fluorophore-tagged reagent,
wherein the reagent source is fluidically coupled to the reaction chamber;
a light source,
wherein the light source emits light having a known wavelength,
wherein the light source is positioned to expose at least a portion of the reaction chamber to the light having a known wavelength;
an optical detector disposed to detect fluorescence emitted from the reaction chamber in response to light emitted from the light source;
a processor configured to communicate with the reversible pump, the reagent source, the light source, and the optical detector, the processor being configured to:
activate the reversible pump to deliver the water sample to the reaction chamber,
prompt delivery of the fluorophore-tagged reagent from the reagent source to the reaction chamber,
activate the light source to expose at least a portion of the reaction chamber to the light having a known wavelength,
obtain a measurement of fluorescence intensity from the optical detector,
determine a concentration of at least one bacteria in the water sample based on the measurement of fluorescence intensity, and
report the concentration of the at least one bacteria in the water sample.

19. A method of simultaneously detecting a concentration of each of a plurality of target organisms in a water sample, the method comprising:
adding one or more tagged reagents to the water sample comprising a plurality of target organisms,
exposing the water sample to light having a known wavelength;
detecting a plurality of light emissions from the water sample;
determining, by a processor, the type and the concentration of each of the plurality of target organisms in the water sample by detecting an intensity for each of the plurality of light emissions from the water sample.

20. The method according to claim 19, wherein each of the plurality of target organisms produces a species-specific byproduct, and
wherein each of the one or more tagged reagents interact with one of the species-specific byproducts to emit a unique light emission from the water sample.

21. A method according to claim 19, wherein the water sample is from a natural environment, and wherein natural ranges of turbidity occurring in the water sample do not impact the ability of the sensor to detect optical signatures of the species-specific by-products interactions with the one or more tagged reagents.

22. A method according to claim 19, wherein each of the plurality of target organisms is selected from the group consisting of E. coli, coliforms, and Enterococcus.

23. A method according to claim 19, wherein the one or more tagged reagents are water soluble and flowable in a liquid medium such that they can be pumped from one area to another.

24. A method according to claim 19, wherein the one or more tagged reagents do not impact the ability of the plurality of target organisms to continue to grow after sampling.

25. A method according to claim 19, wherein the one or more tagged reagents comprise a food source for the target organisms.

26. A method according to claim 19, wherein the one or more tagged reagents have a peak emission separation of at least 50 nm and can be distinguishably detected between 100-1000 nm.

27. A method according to claim 26, wherein the determining step comprises:
distinguishing, by a processor, between the one or more tagged reagents by comparing a plurality of emission peaks.

28. A method according to claim 19, wherein each light emission of the plurality of light emissions changes over time, and
wherein the change of each light emission of the plurality of light emissions over time corresponds to an initial concentration of one of the plurality of target organisms present in the water sample.

29. A method according to claim 19, wherein each light emission of the plurality of light emissions changes over time, and
wherein a change in one of the plurality of light emissions over a time period of less than 6 hours corresponds to an amount of pre-existing by-products of at least one of the plurality of target organisms in the water sample prior to addition of the one or more tagged reagents,
wherein the by-products are selected from the group consisting of metabolic materials and enzymatic materials.

30. A method according to claim 19, wherein each light emission of the plurality of light emissions changes over time, and
wherein a change in one of the plurality of light emissions over a time period of more than 6 hours corresponds to an amount of by-products exudated by at least one of the plurality of target organisms after addition of the one or more tagged reagents,
wherein the by-products are selected from the group consisting of metabolic materials and enzymatic materials.

31. A method according to claim 19, wherein each light emission of the plurality of light emissions changes over time,
wherein an initial rate of change of the plurality of light emissions corresponds to a concentration of by-products of the plurality of target organisms that are present in the water sample, and
wherein the by-products are selected from the group consisting of metabolic materials and enzymatic materials.

33. A method according to claim 19,
wherein the water sample comprises by-products of the plurality of target organisms,
wherein the by-products are selected from the group consisting of metabolic materials and enzymatic materials, and
wherein the concentration of by-products in the water sample corresponds to the concentration of the plurality of target organisms in the water sample.

32. A method according to claim 19, wherein determining the type and the concentration of each of the plurality of target organisms in the water sample is not dependent on growth of the organisms.
